(19) United States
(12) Patent Application Publication
(10) Pub. No.: US 2019/0175142 A1
(43) Pub. Date: Jun. 13, 2019

(54) ULTRASONIC DIAGNOSTIC APPARATUS, MEDICAL IMAGE PROCESSING APPARATUS, AND METHOD FOR CALCULATING PLAQUE SCORE

(71) Applicant: CANON MEDICAL SYSTEMS CORPORATION, Otawara-shi (JP)

(72) Inventors: Katsuyuki Takamatsu, Yaita (JP); Yasunori Ohshima, Yaita (JP); Tomoko Suzuki, Nasushiobara (JP); Taku Muramatsu, Otawara (JP)

(73) Assignee: CANON MEDICAL SYSTEMS CORPORATION, Otawara-shi (JP)

(21) Appl. No.: 16/214,630

(22) Filed: Dec. 10, 2018

(30) Foreign Application Priority Data
Dec. 12, 2017 (JP) 2017-237951

(51) Int. Cl. A61B 8/08

(52) U.S. Cl. CPC A61B 8/0891; A61B 8/5223; A61B 8/461; A61B 8/5246; A61B 8/488

(57) ABSTRACT

The ultrasonic diagnostic apparatus according to a present embodiment includes processing circuitry. The processing circuitry is configured to extract three-dimensional luminal region data out of volume data including a lumen. The processing circuitry is configured to determine a branch plane of the lumen based on the three-dimensional luminal region data. The processing circuitry is configured to divide the lumen based on the branch plane to determine sections. The processing circuitry is configured to estimate a plaque in each section of the sections based on the three-dimensional luminal region data. The processing circuitry is configured to calculate a maximum luminal wall thickness of the each section based on the plaque. The processing circuitry is configured to calculate a plaque score based on the maximum luminal wall thickness of the each section.

### CROSS-REFERENCE TO RELATED APPLICATION

This application is based upon and claims the benefit of priority from Japanese Patent Application No. 2017-237951, filed on Dec. 12, 2017, the entire contents of each of which are incorporated herein by reference.

### FIELD

An embodiment as an aspect of the present invention relates to an ultrasonic diagnostic apparatus, a medical image processing apparatus and a method for calculating a plaque score.

### BACKGROUND

In the medical field, an ultrasonic diagnostic apparatus is used for imaging the inside of a subject using ultrasonic waves generated by multiple transducers (piezoelectric transducers) of an ultrasonic probe. The ultrasonic diagnostic apparatus causes the ultrasonic probe, which is connected to the ultrasonic diagnostic apparatus, to transmit ultrasonic waves into the subject, generates an echo signal based on a reflected wave, and obtains a desired ultrasonic image by image processing.

In the conventional ultrasonic diagnostic apparatus, a B mode image which is the ultrasonic image processed in the B mode based on the echo signal is used in order to grasp the structure of an organ or the like. The B mode image is a so-called black and white image, and is an image expressing a difference in structure depending on a difference in luminance value. In the B mode image, since the reflection state of the ultrasonic waves is different depending on the difference in the tissue, it is possible to clearly express the boundary or the like with respect to the portions having different tissue properties largely.

Arteriosclerosis may cause a plaque. The plaque occurs in the intimal wall of blood vessels. The thickening of the plaque causes the blood vessel to be thinner, or the whole or a part of the plaque is peeled off to be a thrombus and the blood vessel is clogged, causing symptoms such as myocardial infarction. Determination of arteriosclerosis can be performed by carotid pulse echocardiography by the ultrasonic diagnostic apparatus. Plaque scores quantifying the presence or absence of plaque and thickness of blood vessel wall can be calculated based on two dimensional carotid artery region data obtained by carotid pulse echocardiography.

### BRIEF DESCRIPTION OF THE DRAWINGS

FIG. 1 is a schematic diagram showing a configuration of an ultrasonic diagnostic apparatus according to a present embodiment.

FIG. 2 is a block diagram showing functions of the ultrasonic diagnostic apparatus according to the present embodiment.

FIG. 3 is a flowchart showing the operation of the ultrasonic diagnostic apparatus according to the present embodiment.

FIG. 4 is a diagram schematically showing three-dimensional carotid artery region data extracted in the ultrasonic diagnostic apparatus according to the present embodiment.

FIG. 5 is a diagram schematically showing each section of the carotid artery determined in the ultrasonic diagnostic apparatus according to the present embodiment.

FIG. 6 is a flowchart showing the operations of steps ST5 and ST6 in the ultrasonic diagnostic apparatus according to the present embodiment.

FIG. 7 is a diagram for explaining a plaque estimating method in the ultrasonic diagnostic apparatus according to the present embodiment.

FIG. 8 is a diagram for explaining a plaque estimating method in the ultrasonic diagnostic apparatus according to the present embodiment.

Each of FIGS. 9A and 9B is a diagram showing plaques estimated in the ultrasonic diagnostic apparatus according to the present embodiment.

FIG. 10 is a diagram showing a comparative example of FIGS. 9 and 10.

FIG. 11 is a diagram showing a first display example in the ultrasonic diagnostic apparatus according to the present embodiment.

FIG. 12 is a diagram showing a second display example in the ultrasonic diagnostic apparatus according to the present embodiment.

FIG. 13 is a block diagram showing functions of a medical image processing apparatus according to a present embodiment.

### DETAILED DESCRIPTION

An ultrasonic diagnostic apparatus, a medical image processing apparatus and a method for calculating a plaque score according to a present embodiment will be described with reference to the accompanying drawings.

The ultrasonic diagnostic apparatus according to a present embodiment includes processing circuitry. The processing circuitry is configured to execute a volume scan on a region including a lumen to generate volume data including the lumen. The processing circuitry is configured to extract three-dimensional luminal region data out of the volume data including the lumen. The processing circuitry is configured to determine a branch plane of the lumen based on the three-dimensional luminal region data. The processing circuitry is configured to divide the lumen based on the branch plane to determine sections. The processing circuitry is configured to estimate a plaque in each section of the sections based on the three-dimensional luminal region data. The processing circuitry is configured to calculate a maximum luminal wall thickness of the each section based on the plaque. The processing circuitry is configured to calculate a plaque score based on the maximum luminal wall thickness of the each section.

1. Ultrasonic Diagnostic Apparatus

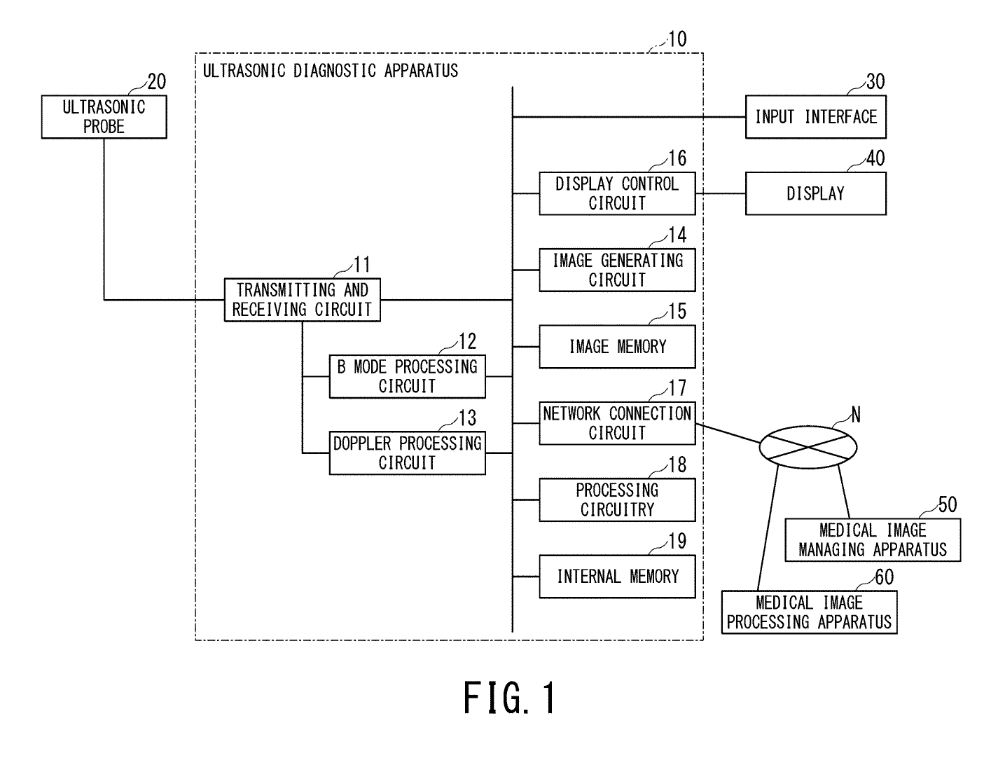

FIG. 1 is a schematic diagram showing a configuration of an ultrasonic diagnostic apparatus according to a present embodiment.

FIG. 1 shows an ultrasonic diagnostic apparatus 10 according to a present embodiment. FIG. 1 shows an ultrasonic probe 20, an input unit (for example, an input interface) 30, and a display unit (for example, a display) 40. It should be noted that a device in which at least one of the ultrasonic probe 20, the input interface 30, and the display 40 is added to the ultrasonic diagnostic apparatus 10 may be referred to as an ultrasonic diagnostic apparatus in some cases. In the following description, a case where all of the ultrasonic probe 20, the input interface 30, and the display 40 are provided outside the ultrasonic diagnostic apparatus 10 will be described.

The ultrasonic diagnostic apparatus 10 includes a transmitting and receiving unit (for example, a transmitting and receiving circuit) 11, a B mode processor (for example, a B mode processing circuit) 12, a Doppler processor (for example, a Doppler processing circuit) 13, an image generator (for example, an image generating circuit) 14, an image storage (for example, an image memory) 15, a display controller (for example, a display control circuit) 16, a network connector (for example, a network connection circuit) 17, a processor (for example, processing circuitry) 18, and a storage (for example, an internal memory) 19. The circuits 11 to 14 are configured by an application specific integrated circuit (ASIC) or the like. However, the present invention is not limited to this case, and all or a part of the functions of the circuits 11 to 14 may be realized by the processing circuitry 18 executing a program.

The transmitting and receiving circuit 11 has a transmitting circuit and a receiving circuit (not shown). Under the control of the processing circuitry 18, the transmitting and receiving circuit 11 controls transmission directivity and reception directivity in transmission and reception of ultrasonic waves. The case where the transmitting and receiving circuit 11 is provided in the ultrasonic diagnostic apparatus 10 will be described, but the transmitting and receiving circuit 11 may be provided in the ultrasonic probe 20, or may be provided in both of the ultrasonic diagnostic apparatus 10 and the ultrasonic probe 20.

The transmitting circuit has a pulse generating circuit, a transmission delay circuit, a pulsar circuit and the like, and supplies a drive signal to ultrasonic transducers. The pulse generating circuit repeatedly generates a rate pulse for forming a transmission ultrasonic wave at a predetermined rate frequency. The transmission delay circuit converges the ultrasonic waves generated from the ultrasonic transducer of the ultrasonic probe 20 into a beam shape, and gives a delay time for each piezoelectric transducer necessary for determining the transmission directivity to each rate pulse generated by the pulse generating circuit. In addition, the pulsar circuit applies a drive pulse to the ultrasonic transducers at a timing based on the rate pulse. The transmission delay circuit arbitrarily adjusts the transmission direction of the ultrasonic beam transmitted from a piezoelectric transducer surface by changing the delay time given to each rate pulse.

The receiving circuit has an amplifier circuit, an A/D (Analog to Digital) converter, an adder, and the like, and receives the echo signal received by the ultrasonic transducers and performs various processes on the echo signal to generate echo data. The amplifier circuit amplifies the echo signal for each channel, and performs gain correction processing. The A/D converter A/D-converts the gain-corrected echo signal, and gives a delay time necessary for determining the reception directivity to the digital data. The adder adds the echo signal processed by the A/D converter to generate echo data. By the addition processing of the adder, the reflection component from the direction corresponding to the reception directivity of the echo signal is emphasized.

Under the control of the processing circuitry 18, the B mode processing circuit 12 receives the echo data from the receiving circuit, performs logarithmic amplification, envelope detection processing and the like, thereby generating data (two-dimensional or three-dimensional data) whose signal intensity is represented by brightness of luminance. This data is generally called B mode data.

Under the control of the processing circuitry 18, the Doppler processing circuit 13 frequency-analyzes the phase information from the echo data from the receiving circuit, and extracts the blood flow or tissue due to the Doppler effect, thereby generating data (two-dimensional or three-dimensional data) obtained by extracting moving state information such as average speed, dispersion, power and the like for multiple points. This data is generally called Doppler data.

Under the control of the processing circuitry 18, the image generating circuit 14 generates an ultrasonic image expressed in a predetermined luminance range as image data based on the echo signal received by the ultrasonic probe 20. For example, the image generating circuit 14 generates a B mode image in which the intensity of the reflected wave is expressed in luminance from the two-dimensional B mode data generated by the B mode processing circuit 12 as the ultrasonic image. Further, the image generating circuit 14 generates, as the ultrasonic image, a color Doppler image representing moving state information from the two-dimensional Doppler data generated by the Doppler processing circuit 13 such as an average velocity image, a dispersed image, a power image, or a combined image thereof.

The image memory 15 includes memory cells in two axial directions per frame, and includes a two-dimensional memory which is a memory having the memory cells for frames. Under the control of the processing circuitry 18, the two-dimensional memory as the image memory 15 stores the ultrasonic image of one frame or the ultrasonic images frames generated by the image generating circuit 14 as two-dimensional image data.

Under the control of the processing circuitry 18, the image generating circuit 14 performs three-dimensional reconstruction on the ultrasonic image arranged in the two-dimensional memory as the image memory 15, if necessary, by interpolation processing, thereby generating an ultrasonic image as volume data in a three-dimensional memory as the image memory 15. As an interpolation processing method, a known technique is used.

The image memory 15 may include a three-dimensional memory which is a memory having memory cells in three axial directions (X-axis, Y-axis, and Z-axis direction). The three-dimensional memory as the image memory 15 stores the ultrasonic image generated by the image generating circuit 14 as volume data under the control of the processing circuitry 18.

The display control circuit 16 includes a graphics processing unit (GPU), a Video RAM (VRAM), and the like. Under the control of the processing circuitry 18, the display control circuit 16 displays the ultrasonic image (for example, a live image), requested for display output from the processing circuitry 18, to the display 40.

The network connection circuit 17 implements various information communication protocols according to the form of the network. In accordance with these various protocols, the network connection circuit 17 connects the ultrasonic diagnostic apparatus 10 and other devices such as the external medical image managing apparatus 50 and the medical image processing apparatus 60. As this connection, electrical connection or the like via an electronic network can be applied. In this embodiment, the electronic network means the whole information communication network using the telecommunication technology, and includes a local area network (LAN) of a wireless/wired hospital core and an internet network, a telephone communication network, an optical fiber communication network, a cable communication network, a satellite communication network, and the like.

Further, the network connection circuit 17 may implement various protocols for non-contact wireless communication. In this case, the ultrasonic diagnostic apparatus 10 can directly exchange data with the ultrasonic probe 20, for example, without going through the network.

The processing circuitry 18 means an ASIC, a programmable logic device, etc. in addition to a dedicated or general purpose central processing unit (CPU), a micro processor unit (MPU), or graphics processing unit (GPU). As the programmable logic device, for example, a simple programmable logic device (SPLD), a complex programmable logic device (CPLD), a field programmable gate array (FPGA).

Further, the processing circuitry 18 may be constituted by a single circuit or a combination of independent circuit elements. In the latter case, the internal memory 19 may be provided individually for each circuit element, or a single internal memory 19 may store programs corresponding to the functions of the circuit elements.

The internal memory 19 is constituted by a semiconductor memory element such as a random access memory (RAM), a flash memory, a hard disk, an optical disk, or the like. The internal memory 19 may be constituted by a portable medium such as a universal serial bus (USB) memory and a digital video disk (DVD). The internal memory 19 stores various processing programs (including an OS (operating system) and the like besides the application program) used in the processing circuitry 18 and data necessary for executing the programs. In addition, the OS may include a graphical user interface (GUI) which allows the operator to frequently use graphics to display information on the display 40 to the operator and can perform basic operations by the input interface 30.

The ultrasonic probe 20 includes microscopic transducers (piezoelectric elements) on the front surface portion, and transmits and receives ultrasonic waves to a region including a scan target, for example, a region including a lumen. Each transducer is an electroacoustic transducer, and has a function of converting electric pulses into ultrasonic pulses at the time of transmission and converting reflected waves to electric signals (reception signals) at the time of reception. The ultrasonic probe 20 is configured to be small and lightweight, and is connected to the ultrasonic diagnostic apparatus 10 via a cable (or wireless communication).

The ultrasonic probe 20 is classified into types such as a linear type, a convex type, a sector type, etc., depending on a difference in scanning system. The ultrasonic probe 20 is classified into a 1D array probe in which transducers are arrayed in a one-dimensional (1D) manner in the azimuth direction, and a 2D array probe in which transducers are arrayed in two dimensions (2D) manner in the azimuth direction and in the elevation direction, depending on the array arrangement dimension. The 1D array probe includes a probe in which a small number of transducers are arranged in the elevation direction.

In this embodiment, when a 3D scan, that is, a volume scan is executed, the 2D array probe having a scan type such as the linear type, the convex type, the sector type, or the like is used as the ultrasonic probe 20. Alternatively, when the volume scan is executed, the 1D probe having a scan type such as the linear type, the convex type, the sector type and the like and having a mechanism that mechanically oscillates in the elevation direction is used as the ultrasonic probe 20. The latter probe is also called a mechanical 4D probe.

In the embodiment, since the volume scan is premised, the 2D array probe or the mechanical 4D probe is adopted as the ultrasonic probe 20. When a region including a carotid artery to be scanned is scanned, it is common to adopt the linear type as the ultrasonic probe 20.

The input interface 30 includes a circuit for inputting a signal from an input device operable by an operator and an input device. The input device may be a trackball, a switch, a mouse, a keyboard, a touch pad for performing an input operation by touching an operation surface, a touch screen in which a display screen and a touch pad are integrated, a non-contact input circuit using an optical sensor, an audio input circuit, and the like. When the input device is operated by the operator, the input interface 30 generates an input signal corresponding to the operation and outputs it to the processing circuitry 18.

The display 40 is constituted by a general display output device such as a liquid crystal display or an organic light emitting diode (OLED) display. The display 40 displays various kinds of information under the control of the processing circuitry 18.

FIG. 1 shows the medical image managing apparatus 50 and the medical image processing apparatus 60 which are external devices of the ultrasonic diagnostic apparatus 10. The medical image managing apparatus 50 is, for example, a digital imaging and communications in medicine (DICOM) server, and is connected to a device such as the ultrasonic diagnostic apparatus 10 so that data can be transmitted and received via the network N. The medical image managing apparatus 50 manages a medical image such as an ultrasonic image generated by the ultrasonic diagnostic apparatus 10 as a DICOM file.

The medical image processing apparatus 60 is connected to devices such as the ultrasonic diagnostic apparatus 10 and the medical image management apparatus 50 so that data is transmitted and received via the network N. An Example of the medical image processing apparatus 60 includes a workstation that performs various image processing on the ultrasonic image generated by the ultrasonic diagnostic apparatus 10 and a portable information processing terminal such as a tablet terminal. It should be noted that the medical image processing apparatus 60 is an offline apparatus and may be an apparatus capable of reading an ultrasonic image generated by the ultrasonic diagnostic apparatus 10 via a portable storage medium.

Subsequently, functions of the ultrasonic diagnostic apparatus 10 will be described.

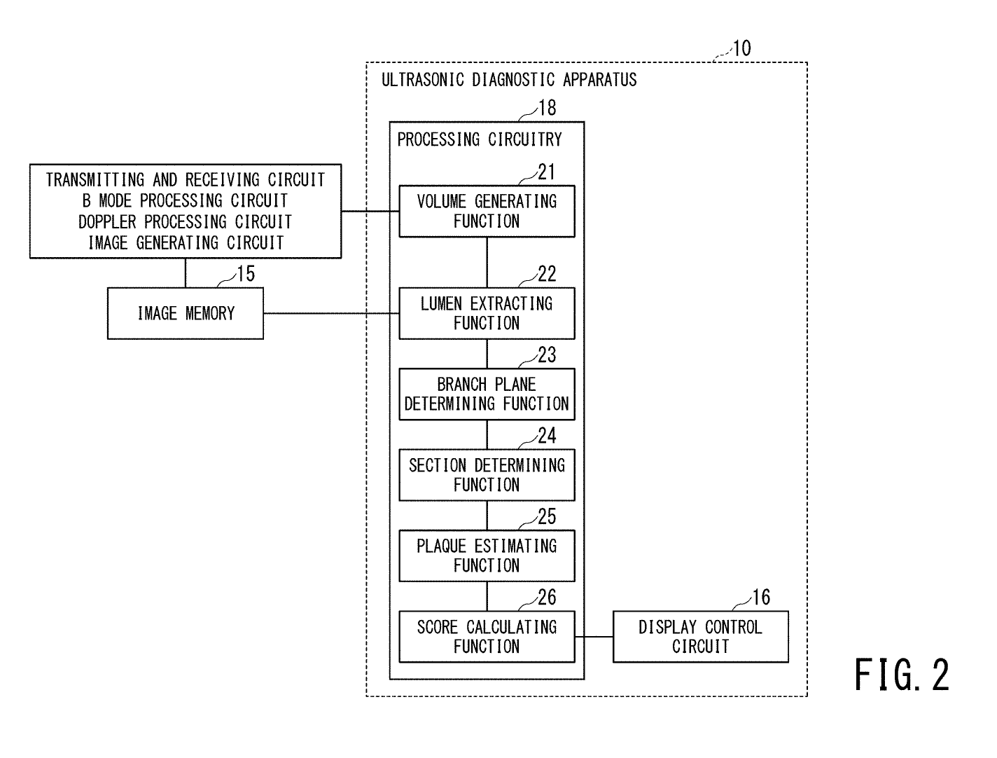

FIG. 2 is a block diagram showing functions of the ultrasonic diagnostic apparatus 10.

The processing circuitry 18 reads out and executes a program stored in the internal memory 19 or directly incorporated in the processing circuitry 18, thereby realizing a volume generating unit (for example, a volume generating function) 21, a lumen extracting unit (for example, a lumen extracting function) 22, a branch plane determining unit (for example, a branch plane determining function) 23, a section determining unit (for example, a section determining function) 24, a plaque estimating unit (for example, a plaque estimating function) 25, and a score calculating unit (for example, a score calculating function) 26. Hereinafter, a case where the functions 21 to 26 function as software will be described as an example. All or a part of the functions 21 to 26 may be provided as a circuit or the like of ASIC etc. in the ultrasonic diagnostic apparatus 10.

The volume generating function 21 is a function of comprehensively controlling, in accordance with the input from the input interface 30, the transmitting and receiving circuit 11 and the like to execute a volume scan on a region including a luminal of a patient, thereby generating volume data including the luminal. The volume generating function 21 stores the volume data in the image memory 15.

The lumen extracting function 22 is a function of acquiring the volume data including the lumen out of the image memory 15 and extracting three-dimensional luminal region data from the volume data including the lumen.

The branch plane determining function 23 is a function of determining a branch plane of the lumen based on the three-dimensional luminal region data extracted by the lumen extracting function 22. The branch plane determining function 23 detects a bulging point of the branch of the lumen based on the three-dimensional luminal region data and determines a plane including the bulging point of the lumen.

The section determining function 24 is a function of determining sections for dividing the lumen based on the three-dimensional luminal region data extracted by the lumen extracting function 22 and on the branch plane determined by the branch plane determining function 23. The section determining function 24 calculates, for example, four divided planes with reference to the branch plane detected by the branch plane determining function 23, and divides the lumen into four sections in three dimensions.

The plaque estimating function 25 is a function of estimating a plaque in each section of the sections determined by the section determining function 24 based on the three-dimensional luminal region data extracted by the lumen extracting function 22. For example, the plaque estimating function 25 obtains a displacement of the luminal wall thickness of each section based on the three-dimensional luminal region data, and estimates the plaque by comparing the luminal wall thickness associated with a vertex (peak) in the displacement with a threshold value. The plaque estimating function 25 calculates the luminal wall thickness, for example, an intima-media thickness (IMT), and estimates a portion having the IMT equal to or larger than the threshold as the plaque. The IMT indicates the thickness including a tunica intima and a media constituting the surface layer of the carotid artery lumen side.

The score calculating function 26 is a function of calculating the maximum IMT of each section based on the plaque estimated by the plaque estimating function 25 and calculating a plaque score based on the maximum IMT of each section.

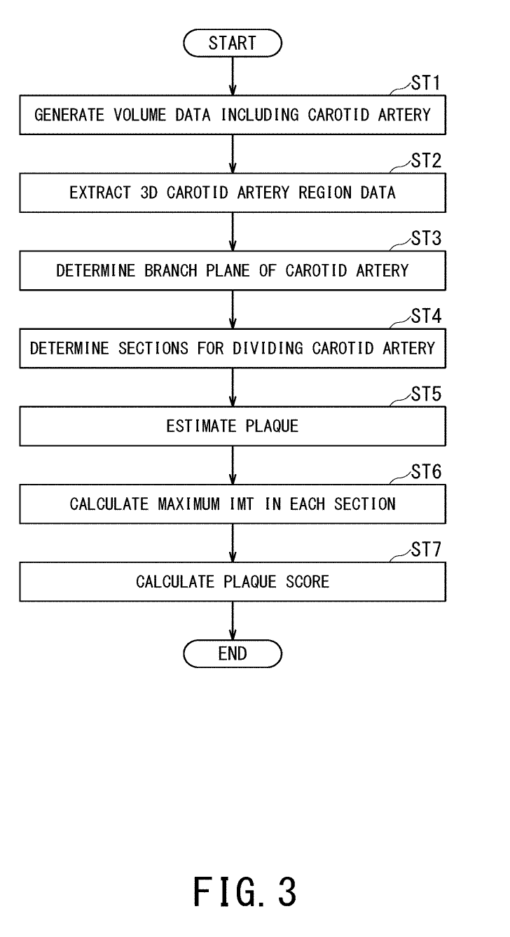

Details of the functions of the functions 21 to 26 will be described later with reference to FIGS. 3 to 11.

Subsequently, the operation of the ultrasonic diagnostic apparatus 10 will be described.

FIG. 3 is a flowchart showing the operation of the ultrasonic diagnostic apparatus 10. In FIG. 3, the reference numerals assigned “ST” with numerals indicate the respective steps of the flowchart.

The volume generation function 21 comprehensively controls, in accordance with a scan starting instruction from the operator via the input interface 30, the transmitting and receiving circuit 11 and the like to execute a volume scan for a region including a lumen, for example, a carotid artery, thereby generating volume data including the carotid artery (step ST1). Hereinafter, the case where the lumen is the carotid artery will be described, but it is not limited to that case. For example, the lumen may be a blood vessel such as an arm or a lower limb liable to cause stenosis.

When the volume scan is executed in step ST1, a live image based on the volume data is generated and displayed. The volume data including the carotid artery generated in step ST1 is stored in the image memory 15.

The lumen extracting function 22 acquires the volume data including the carotid artery from the image memory 15 and extracts three-dimensional carotid artery region data as luminal region data out of the volume data including the carotid artery (step ST2).

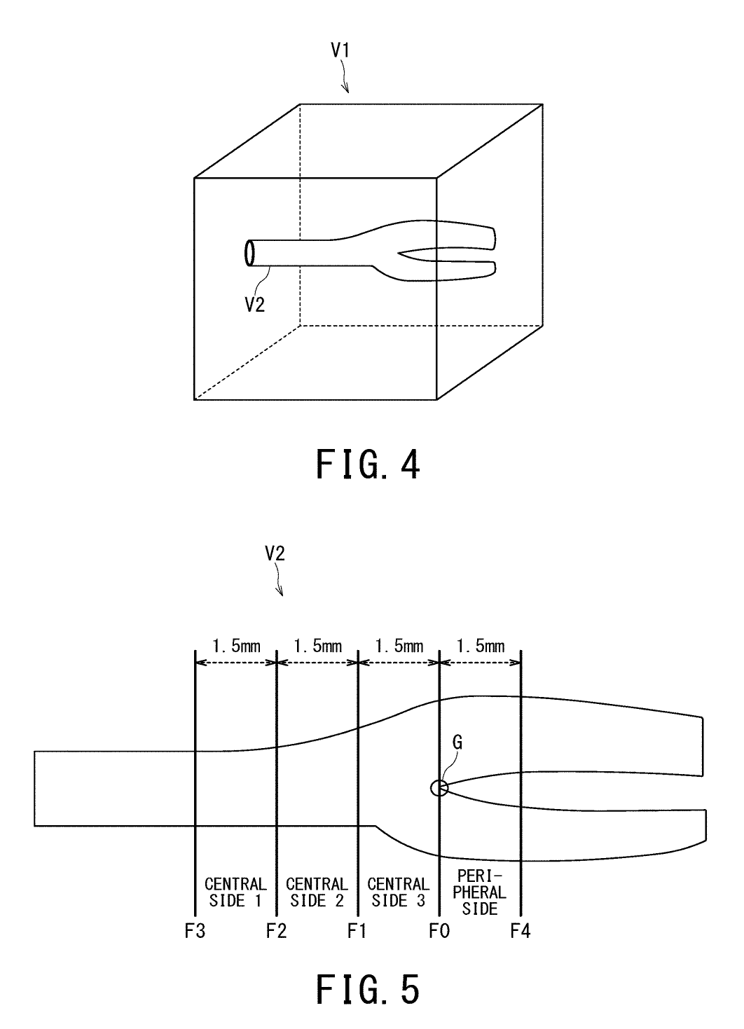

FIG. 4 is a diagram schematically showing three-dimensional carotid artery region data extracted in the ultrasonic diagnostic apparatus 10.

FIG. 4 shows volume data V1 including the carotid artery and the three-dimensional carotid artery region data V2 included in the volume data V1. Three-dimensional carotid artery region data V2 is extracted from the volume data V1 including the carotid artery.

Returning to the explanation of FIG. 3, the branch plane determining function 23 determines the branch plane of the carotid artery based on the three-dimensional carotid artery region data extracted in step ST2 (step ST3). The branch plane determining function 23 detects a bulging point of an internal carotid artery and an external carotid artery of the carotid artery based on the three dimensional carotid artery region data, and determines a plane including the bulging point as the branch plane. An example of the determined branch plane is shown in FIG. 5 to be described later.

The section determining function 24 divides the carotid artery based on the three-dimensional carotid artery region data extracted in step ST2 and the branch plane determined in step ST3, and determines sections (step ST4).

FIG. 5 is a diagram schematically showing each section of the carotid artery determined in the ultrasonic diagnostic apparatus 10.

FIG. 5 shows three-dimensional carotid artery region data V2. In addition, FIG. 5 shows a branch plane F0 set on the three-dimensional carotid artery region data V2 and dividing planes F1 to F4 determined based on the branch plane F0. The branch plane F0 may be determined according to the operation by the operator with reference to a bulging point G of the internal carotid artery and the external carotid artery of the carotid artery or may be determined as the tangential plane of the bulging point G. The dividing planes F1 to F4 are determined so as to be parallel to the branch plane F0. Further, the dividing plane F1 is located at a position distant from the branch plane F0 by a certain value, for example, 1.5 [cm], and the dividing plane F2 is located at a position 1.5 [cm] away from the dividing plane F1 toward the central side, and the dividing plane F3 is located at a position 1.5 [cm] away from the dividing plane F2 toward the central side. On the other hand, the dividing plane F4 is located at a position separated by 1.5 [cm] from the branch plane F0 to the peripheral side.

A space between the dividing plane F3 and the dividing plane F2 is called “central side 1” of the carotid artery, a space between the dividing plane F2 and the dividing plane F1 is called “central side 2” of the carotid artery, a dividing plane F1 and the branch plane F0 is called “central side 3” of the carotid artery. Also, a space between the branch plane F0 and the dividing plane F4 is called “peripheral side” of the carotid artery.

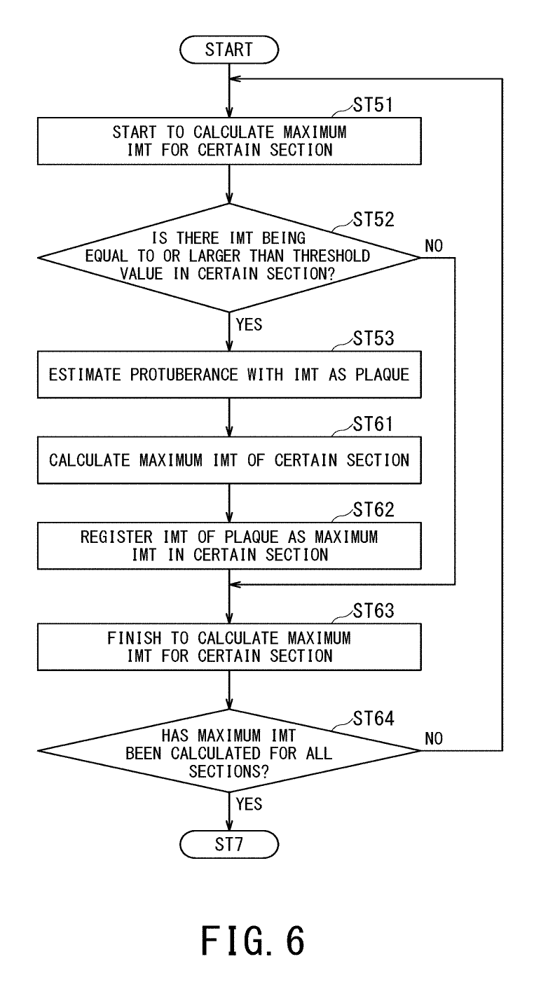

Returning to the explanation of FIG. 3, based on the three-dimensional carotid artery region data extracted in step ST2, the plaque estimating function 25 calculates an IMT of the lumen in each section of the sections determined in step ST4, and estimates a portion having a certain IMT or more as a plaque (step ST5). The score calculating function 26 determines the maximum IMT in each section based on the plaque estimated in step ST5 (step ST6). Details of steps ST5 and ST6 will be described with reference to FIG. 6.

FIG. 6 is a flowchart showing the operations of steps ST5 and ST6 in the ultrasonic diagnostic apparatus 10. In FIG. 6, the reference numerals assigned “ST” with numerals indicate the respective steps of the flowchart.

The plaque estimating function 25 starts calculating the maximum IMT for a certain section out of the four sections of the carotid artery (step ST51). The plaque estimating function 25 obtains a displacement of the IMT in the relevant section based on the three-dimensional luminal region data extracted by step ST2 (shown in FIG. 3), and determined whether there is an IMT relating to a vertex (peak) in the displacement and being equal to or larger than the threshold value, for example, 1.1 [mm] or more (step ST52).

For example, the plaque estimating function 25 calculates slice planes C (shown in FIG. 9A) parallel to the branch plane F0 in the section and sequentially calculates the IMT on the slice planes. Since the diameter of the carotid artery is relatively small and the degree of bending of the carotid artery at the bifurcation is also relatively small, the thickness direction of the IMT can be considered to be on the slice plane. Alternatively, the plaque estimating function 25 may calculate the slice planes orthogonal to the core line direction of the carotid artery in the section, and sequentially calculate the IMTs on the slice planes.

If it is determined as “YES” in step ST52, that is, if it is determined that there is the IMT relating to the vertex in the displacement and 1.1 [mm] or more, the plaque estimating function 25 estimates a protuberance with the IMT as one or multiple plaques (step ST53).

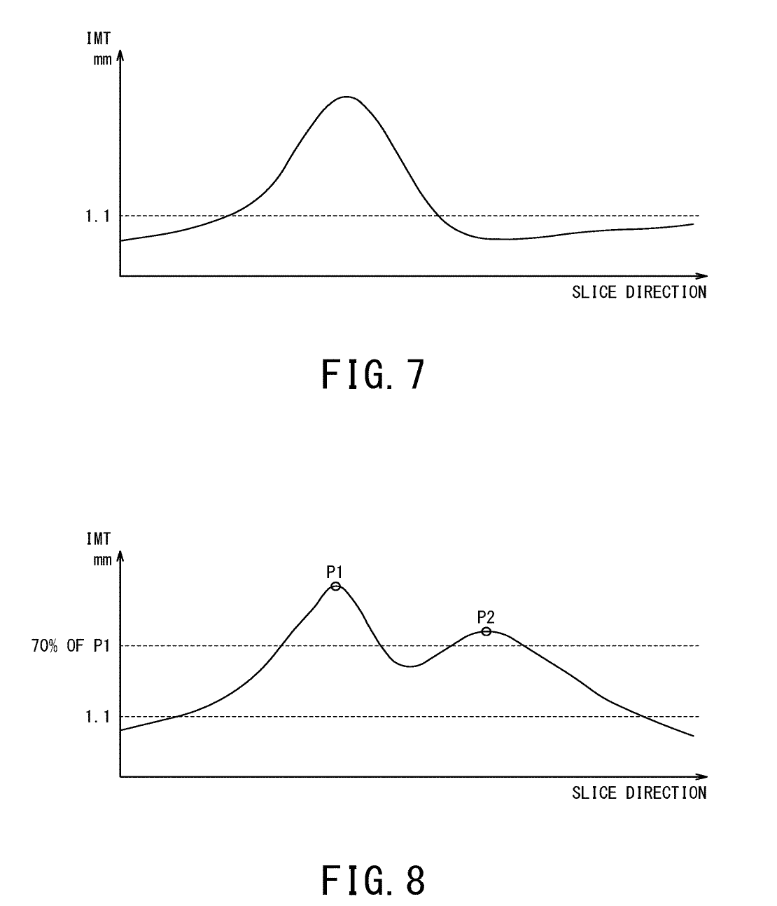

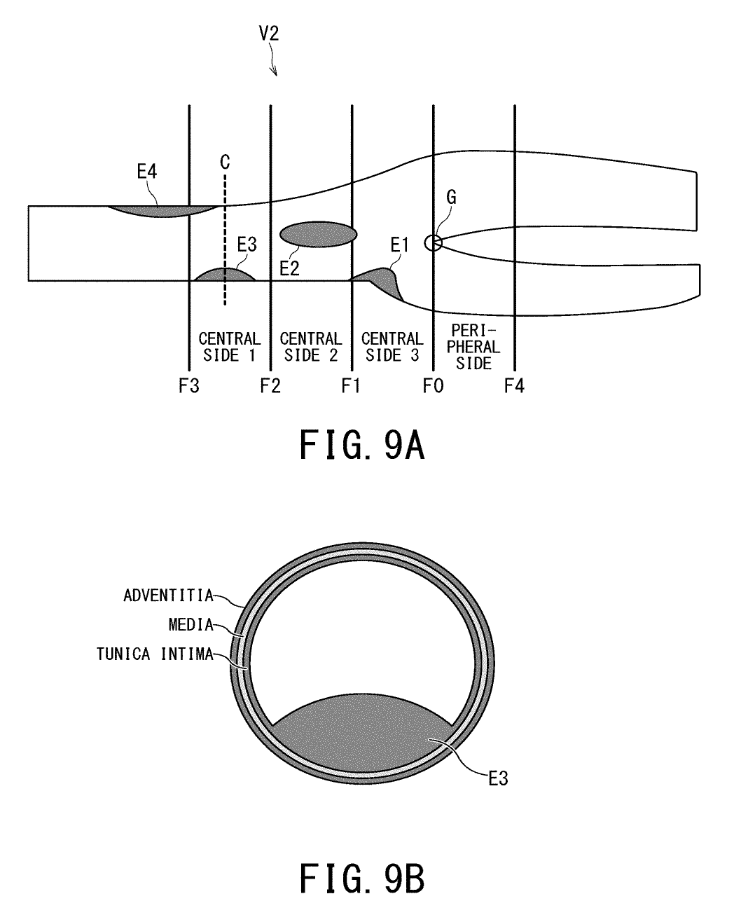

Each of FIGS. 7 and 8 is a diagram for explaining a plaque estimating method in the ultrasonic diagnostic apparatus 10. Each of FIGS. 7 and 8 shows the displacement of the IMT relative to the arrangement direction (i.e. the slice direction) of slice planes parallel to the branch plane F0 (shown in FIG. 5). Each of FIGS. 7 and 8 shows a displacement of the IMT of the carotid artery in slice planes, that is, two-dimensional displacement, but the displacement of the IMT of the carotid artery is obtained as a three-dimensional one.

As shown in FIG. 7, when there is one vertex of the curve showing the displacement of the IMT at a position of 1.1 [mm] or more, the protuberance including the vertex is estimated as a plaque.

As shown in FIG. 8, there are vertices of the curve showing the displacement of the IMT, and it may be difficult to determine whether it is one plaque or two plaques. In this case, if the minimum IMT between the vertex P1 and the vertex P2 drops to a certain percentage with respect to the IMT related to the maximum vertex P1, it is determined to be two plaques. For example, the certain percentage is 70% of the IMT associated with the largest vertex P1. On the other hand, if the IMT between the vertex P1 and the vertex P2 does not drop to less than 70% of the IMT related to the maximum vertex P1, it is determined to be one plaque.

Returning to the explanation of FIG. 6, the score calculating function 26 calculates the maximum IMT of the section based on the plaque estimated in step ST53 (step ST61). The score calculating function 26 registers the maximum IMT of the section calculated in step ST61 (step ST62), and ends the calculation of the maximum IMT for the section started in step ST51 (step ST63).

The score calculating function 26 determines whether or not the maximum IMT has been calculated for all the sections (step ST64). If it is determined as “NO” in step ST64, that is, if it is determined that the maximum IMT has not been calculated for all the sections, the plaque estimating function 25 calculates the maximum IMT for the next section out of the sections of the carotid artery (step ST51).

On the other hand, if it is determined as “YES” in step ST64, that is, if it is determined that the maximum IMT has been calculated for all the sections, the process proceeds to step ST7 in FIG. 3.

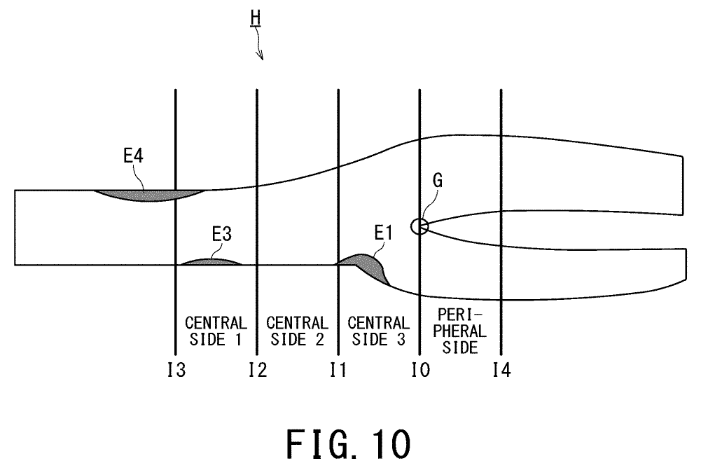

Each of FIGS. 9A and 9B is a diagram showing plaques estimated in the ultrasonic diagnostic apparatus 10. FIG. 10 is a diagram showing a comparative example of FIGS. 9 and 9B.

FIG. 9A shows three-dimensional carotid artery region data V2, a branch plane F0, dividing planes F1 to F4, and plaques E1 to E4 in the carotid artery. Since the carotid artery region data V2 is three-dimensional data, it is possible to estimate all the plaques in each section, including the plaque E2 present on the side wall. FIG. 9B is a view showing a cross section C of FIG. 9A.

On the other hand, FIG. 10 shows two-dimensional carotid artery region data H, branch line I0, dividing lines I1 to I4, and plaque E1, E3 and E4 in the carotid artery as cross-sectional data. On the branch line I0, a bulging point G between the internal carotid artery and the external carotid artery of the carotid artery is detected based on the two-dimensional carotid artery region data H, as the result, a line including the bulging point G is determined as the branch line I0. The dividing lines I1 to I4 divide the carotid artery into four sections two-dimensionally by calculating four dividing lines based on the branch line I0 based on the two-dimensional carotid artery region data H.

When using the two-dimensional carotid artery region data H shown in FIG. 10, it is impossible to estimate all the plaques within the four sections as compared with the case of using the three-dimensional carotid artery region data V2 shown in FIG. 9A. For example, since the two-dimensional carotid artery region data H shown in FIG. 10 is sectional data, it is impossible to estimate plaque E2 on the side of the carotid artery shown in FIG. 9A. Further, when using the two-dimensional carotid artery region data H shown in FIG. 10, it is impossible to accurately calculate the IMT of the plaque as compared with the case of using the three-dimensional carotid artery region data V2 shown in FIG. 9A. For example, since the two-dimensional carotid artery region data H shown in FIG. 10 is section data, the IMT of plaques E1 and E3 to be calculated is not always the maximum value. In order to solve such a problem, it is necessary to estimate the plaque based on two-dimensional carotid artery region data on multiple different cross sections, but in that case, the process is complicated.

On the other hand, when the three-dimensional carotid artery region data V2 shown in FIG. 9A is used, it is possible to estimate all the plaques in each section of the carotid artery. Further, when using the three-dimensional carotid artery region data V2 shown in FIG. 9A, it is possible to accurately calculate the IMT of the plaque. When there are plaques (for example, plaque E4) spanning two sections, the plaque is estimated as being in the section where the maximum IMT portion exists.

Returning to the explanation of FIG. 3, the score calculating function 26 calculates a plaque score based on the maximum IMT of each section calculated in step ST6 (step ST7). The score calculating function 26 calculates at least one of the plaque score of the left carotid artery, the plaque score of the right carotid artery, and the plaque score of the carotid arteries on both sides. The plaque score for the right (or left) carotid artery is the sum of the maximum IMT for four sections. The plaque score for the carotid arteries on both sides is the sum of the maximum IMT for the left and right 8 sections. It should be noted that the score calculating function 26 also calculates the number of plaques (Plaque Number) as the sum of the number of plaques.

The score calculating function 26 generates a cross-sectional image of a cross section showing the maximum IMT of each section, and displays four cross-sectional images corresponding to the four sections on the display 40.

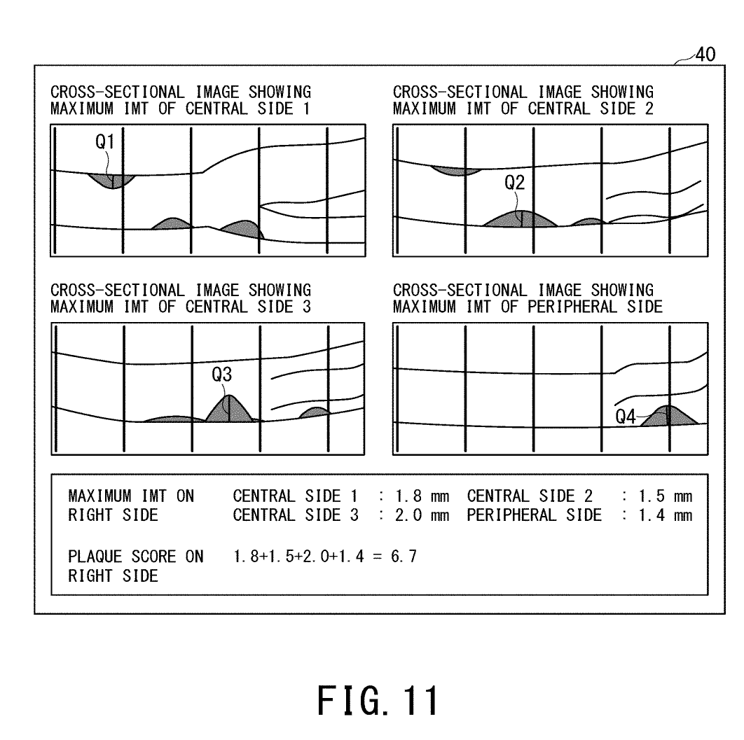

FIG. 11 is a diagram showing a first display example in the ultrasonic diagnostic apparatus 10.

FIG. 11 shows four cross-sectional image data (cross-sectional images) in the case where the whole of the three-dimensional carotid artery region data is cut by the number of sections (four sections). The upper left part of FIG. 11 is a cross-sectional image in the case where the whole of the three-dimensional carotid artery region is cut along the cross section showing the maximum IMT_Q1 of the central side 1. That is, this cross-sectional image relates to a cross section including the thickness direction of the maximum IMT_Q1 of the central side 1. The upper right part of FIG. 11 is a cross-sectional image in the case where the whole of the three-dimensional carotid artery region is cut along the cross section showing the maximum IMT_Q2 of the central side 2.

The lower left part of FIG. 11 is a cross-sectional image in the case where the whole of the three-dimensional carotid artery region is cut along the cross section showing the maximum IMT_Q3 of the central side 3. The lower right part of FIG. 11 is a cross-sectional image in the case where the whole of the three-dimensional carotid artery region is cut along the cross section showing the maximum IMT_Q4 of the peripheral side.

As shown in FIG. 11, by displaying four cross-sectional images when the whole of the carotid artery region is cut with four sections, it is possible for the operator to visually recognize the maximum IMT_Q1 to the maximum IMT_Q4 in the four sections.

Further, the score calculating function 26 may display, as the plaque score, at least one of a plaque score related to the left carotid artery, a plaque score related to the right side carotid artery, and a plaque score related to the carotid arteries on both sides, together with the four cross-sectional images or the four cross-sectional images. The plaque score may be displayed on a measurement display area (MDA), a worksheet, or a report on the display screen. In FIG. 11, plaque scores related to the carotid artery on the right side are displayed together with the top four cross-sectional images.

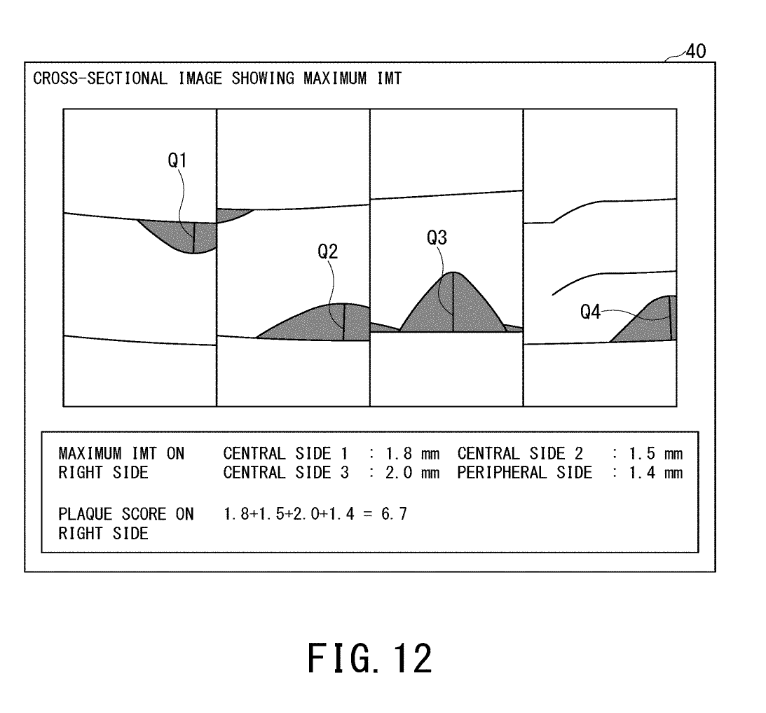

FIG. 12 is a diagram showing a second display example in the ultrasonic diagnostic apparatus 10.

FIG. 12 shows four cross-sectional image data (cross-sectional images) in the case where four sections of three-dimensional carotid artery region data are cut with four sections. The left end portion in FIG. 12 is a cross-sectional image in the case where the central side 1 of the three-dimensional carotid artery region is cut along the cross section showing the maximum IMT_Q1 of the central side 1. The second part from the left in FIG. 12 is a cross-sectional image in the case where the central side 2 of the three-dimensional carotid artery region is cut along the section showing the maximum IMT_Q2 of the central side 2. The third part from the left in FIG. 12 is a cross-sectional image in the case where the central side 3 of the three-dimensional carotid artery region is cut along the cross section showing the maximum IMT_Q3 of the central side 3. The right end portion in FIG. 12 is a cross-sectional image in the case where the peripheral side of the three-dimensional carotid artery region is cut along the cross section showing the maximum IMT_Q4 on the peripheral side.

As shown in FIG. 12, by displaying four cross-sectional images when the four sections of the carotid artery region are cut, it is possible for the operator to visually recognize the maximum IMT_Q1 to the maximum IMT_Q4 in the four sections.

According to the ultrasonic diagnostic apparatus 10, it is possible to present an accurate and sensitive plaque score to the operator because a plaque is estimated based on three-dimensional luminal region data (e.g. carotid artery region data V2).

2. Medical Image Processing Apparatus

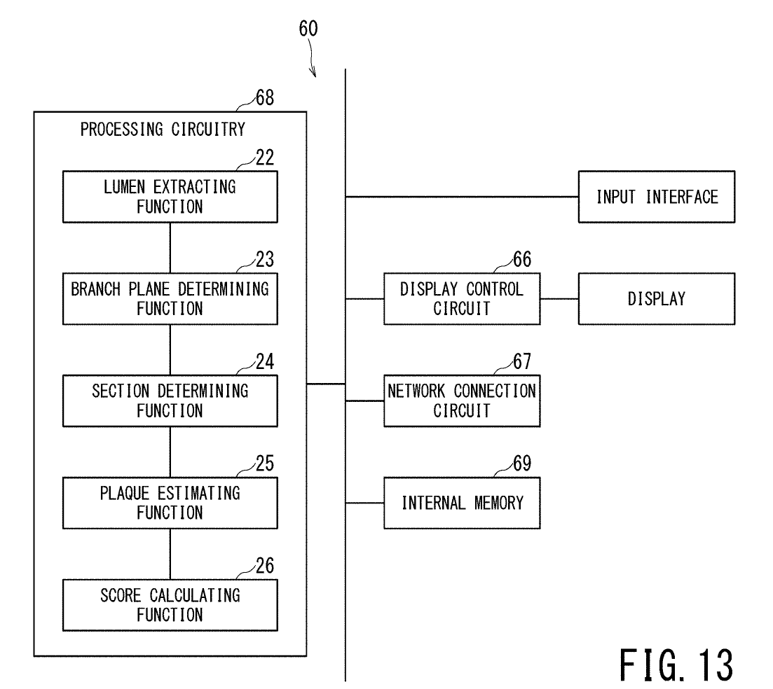

FIG. 13 is a block diagram showing functions of a medical image processing apparatus according to a present embodiment.

FIG. 13 shows a medical image processing apparatus 60 according to an embodiment shown in FIG. 1. The medical image processing apparatus 60 includes a display controller (for example, a display control circuit) 66, a network connector (for example, a network connection circuit) 67, a processor (for example, processing circuitry) 68 and a storage (for example, an internal memory) 69. The medical image processing apparatus 60 may include an input interface and a display.

The configurations of the display control circuit 66, the network connection circuit 67, the processing circuitry 68, and the internal memory 69 are equivalent to the display control circuit 16, the network connection circuit 17, the processing circuitry 18, and the internal memory 19 shown in FIG. 1, respectively, so their explanation is omitted.

The processing circuitry 68 reads out and executes a program stored in the internal memory 69 or directly incorporated in the processing circuitry 68, thereby realizing the lumen extracting function 22, the branch plane determining function 23, the section determining function 24, the plaque estimating function 25, and the score calculating function 26. In FIG. 13, the same members as those shown in FIG. 2 are denoted by the same reference numerals, and description thereof is omitted.

It is possible for the medical image processing apparatus 60 to acquire the ultrasonic image generated by the ultrasonic diagnostic apparatus 10 via the network connection circuit 67 or via a portable storage medium.

The details of the functions 22 to 26 have been described with reference to FIGS. 3 to 12, so that the description thereof will be omitted.

According to the medical image processing apparatus 60, it is possible to present an accurate and sensitive plaque score to the operator because a plaque is estimated based on three-dimensional luminal region data (e.g. carotid artery region data V2).

According to at least one of the embodiments described above, it is possible to present an accurate and sensitive plaque score to the operator.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising: processing circuitry configured to
execute a volume scan on a region including a lumen to generate volume data including the lumen,
extract three-dimensional luminal region data out of the volume data including the lumen,
determine a branch plane of the lumen based on the three-dimensional luminal region data,
divide the lumen based on the branch plane to determine sections,
estimate a plaque in each section of the sections based on the three-dimensional luminal region data,
calculate a maximum luminal wall thickness of the each section based on the plaque, and
calculate a plaque score based on the maximum luminal wall thickness of the each section.

2. The ultrasonic diagnostic apparatus according to claim 1, wherein
the processing circuitry is configured to obtain a displacement of the luminal wall thickness of the each section based on the three-dimensional luminal region data, and to compare the luminal wall thickness relative to a vertex in the displacement with a threshold value, thereby estimating the plaque.

3. The ultrasonic diagnostic apparatus according to claim 1, wherein
the processing circuitry is configured to
generate, as the volume data including the lumen, volume data including a carotid artery, and
extract, as the three-dimensional luminal region data, three-dimensional carotid artery region data out of the volume data including the carotid artery.

4. The ultrasonic diagnostic apparatus according to claim 3, wherein
the processing circuitry is configured to
detect a bulging point of an internal carotid artery and an external carotid artery of the carotid artery based on the three-dimensional carotid artery region data, and
determine a plane including the bulging point as the branch plane.

5. The ultrasonic diagnostic apparatus according to claim 4, wherein
the processing circuitry is configured to determine a tangent plane of the bulging point as the branch plane based on the three-dimensional carotid artery region data.

6. The ultrasonic diagnostic apparatus according to claim 3, wherein
the processing circuitry is configured to calculate at least one of a plaque score of a left carotid artery, a plaque score of a right carotid artery, and a plaque score of the carotid arteries on both sides as the plaque score.

7. The ultrasonic diagnostic apparatus according to claim 1, wherein
the processing circuitry is configured to
calculate a maximum intima-media thickness (IMT) of each section based on the plaque, and
calculate the plaque score based on the maximum IMT of each section.

8. The ultrasonic diagnostic apparatus according to claim 1, wherein
the processing circuitry is configured to display the plaque score on a display.

9. The ultrasonic diagnostic apparatus according to claim 1, wherein
the processing circuitry is configured to
cut a whole of the three-dimensional luminal region data with cross sections corresponding to a number of the sections, thereby generating multiple sectional image data, and
display the multiple cross-sectional image data on a display.

10. The ultrasonic diagnostic apparatus according to claim 1, wherein
the processing circuitry is configured to
cut the sections of the three-dimensional luminal region data with predetermined cross sections, respectively, thereby generating multiple cross-sectional image data corresponding to a number of the sections, and
display the multiple cross-sectional image data on a display.

11. A medical image processing apparatus comprising: processing circuitry configured to
determine a branch plane of a lumen based on three-dimensional luminal region data extracted out of volume data including the lumen,
divide the lumen based on the branch plane to determine sections,
estimate a plaque in each section of the sections based on the three-dimensional luminal region data,
calculate a maximum luminal wall thickness of the each section based on the plaque, and
calculate a plaque score based on the maximum luminal wall thickness of the each section.

12. A method for calculating a plaque score comprising:
determining a branch plane of a lumen based on three-dimensional luminal region data extracted out of volume data including the lumen,
dividing the lumen based on the branch plane to determine sections,
estimating a plaque in each section of the sections based on the three-dimensional luminal region data,
calculating a maximum luminal wall thickness of the each section based on the plaque, and
calculating a plaque score based on the maximum luminal wall thickness of the each section.
